(19) United States
(12) Patent Application Publication
(10) Pub. No.: US 2017/0172790 A1
(43) Pub. Date: Jun. 22, 2017

(54) TEMPERATURE MANAGEMENT AND PHASE CHANGE ENERGY SYSTEM FOR STORAGE AND THERAPEUTIC APPLICATIONS

(71) Applicants: Najih Naser, Cary, NC (US); Amna N. Naser, Cary, NC (US); Abdel-Rahman N. Naser, Cary, NC (US)

(72) Inventors: Najih Naser, Cary, NC (US); Amna N. Naser, Cary, NC (US); Abdel-Rahman N. Naser, Cary, NC (US)

(21) Appl. No.: 15/375,158

(22) Filed: Dec. 11, 2016

Related U.S. Application Data
(63) Continuation of application No. 62/266,563, filed on Dec. 11, 2015.

(51) Int. Cl. A61F 7/08; F25D 3/08; B65D 81/38; F28D 20/02; A61F 7/10; B65D 81/18

(52) U.S. Cl. CPC A61F 7/08; A61F 7/10; B65D 81/18; B65D 81/382; F28D 20/02; F25D 3/08; A61F 2007/0095

(57) ABSTRACT

This invention provides a flexible and dynamic energy storage and transfer device for safe storage and transport of temperature sensitive products and for therapeutic uses. The portable device includes interactive chambers containing energy receptor materials with thermal holding capacity for cooling and heating purposes. Various chambers may be included to provide varying temperature zones. According to yet another aspect of the invention, a secondary layer is used to respond to environmental temperatures including ambient and exhaustive storage temperatures to extend the energy exchange period and sustain a thermal therapeutic dose toward targeted area.

### CROSS REFERENCES OF RELATED APPLICATION

This patent application claims the benefit of U.S. Provisional Patent Application No. 62/266,563, filed on Dec. 11, 2015, the specifications of which are entirely incorporated.

### FIELD OF THE DISCLOSURE

This disclosure relates to a dynamic and modular enclosure apparatus with phase change energy storage receptors and applications thereof. In particular it provides a multifunctional design and capability for the containment and movement of PCM energy receptor materials and to provide temperature protection and therapeutic cooling or heating zones.

### BACKGROUND

The use of preventive measures and protective materials including cold and hot packs has significantly increased in recent years. A variety of devices and containers are used to protect temperature sensitive materials such as food and medicine, as well as therapeutically to release muscle stress and minimize bruises of affected areas due to stress or injuries.

These enclosures, energy storage wrap and pads are important to preserve thermally labile products from spoilage and loss of activity. Some heavy and bulky cold energy packs and containers were developed by freezing water solutions in a bag or container for use in shipping and cooling tasks. A variety of insulated containers and cooling packs were also suggested as described in U.S. Pat. No. 5,417,082, U.S. Pat. No. 5,441,170 and U.S. Pat. No. 6,822,198 B2.

In addition to keeping a cool environment, some products must be maintained at a narrow temperature range and or must be protected from freezing. This applies to a variety of environmentally sensitive goods such as food, medicine, biological specimens and the like. Various methods were suggested including multi-wall containers and phase change energy material solutions. U.S. Pat. No. 7,516,600; U.S. 2013/0255306 Al and U.S. Pat. No. 8,600,903 B2. Other concepts were described to help in providing a therapeutic relief for joints and tissue areas. U.S. Pat. No. 7,179,281 B2, and U.S. Pat. No. 8,226,699 B2.

The limited configurations and geometry of current products may still provide cooling however, they suffer from inflexibility, extra bulky volume and mass which adds extra shipping cost and occupy warehouse space in addition to large waste disposal and impact on the environment. Furthermore, it suffer from disadvantages, such as extreme solid state feel, single temperature zone, low contact surface area, extreme discomfort in cold overdosed area, made with synthetic ingredients and irritant chemicals if packaging is punctured or spilled, low versatility, and difficulty of use. Improved temperature management devices and therapeutic thermal energy storage and delivery enclosure systems are therefore desired. The present disclosure offers a universal and dynamic apparatus for temperature management and energy delivery.

### SUMMARY OF THE INVENTION

A primary objective of the present patent is to provide a new configuration of an interactive enclosure and phase change composition holding media matrix for rapid thermal energy response and enhanced therapeutic energy transmission. In one aspect, the enclosure systems are described herein which, in some embodiments, offer one or more advantages over prior thermal energy exchange systems.

In some embodiments, for example, a progressive thermal energy transfer enclosure described herein is a self-contained system that can be instantly formatted to fit a flat surface or wrapped and formed around three-dimensional surfaces for a more intimate and efficient coverage. Further, in some embodiments, a thermal energy exchange system described herein is spherical, planar, modular, compartmentalized serially and/or in parallel as portable apparatus and modularly interchangeable.

Moreover, in some embodiments, progressive chambers for thermal energy storage and transfer described herein can provide a consortium of shapes and thicknesses to create thin layer pads or transform to make a thick thermal storage pad in one of the compartments by displacing the energy receptor media through the channels between the chambers, thereby improving versatility and efficiency for a greater energy transition and healing power. Additionally, a thermal energy receptor media described herein comprises a subzero freezing point formulation as well as higher temperatures. The phase change and energy receptor material are efficient in storing and discharging energy, of high collective energy properties, safe, easily available and is self-contained for easy handling.

In other embodiments, phase change material charging and discharging of energy is improved by the integration of a media matrix to contain the energy storage composition and increase the rate of phase transition. This enhances a reversible phase change transition and rapid crystallization during the energy discharge step. In one embodiment, a therapeutic energy pad to provide body cooling effect may be produced by the addition of a three dimensional media matrix with high absorption affinity and large surface area. The media's network allows for the containment of various compositions including organic and inorganic phase change materials and stabilizers thus enhancing the thermal conductivity and rapid response to changes in the environment temperatures. The active channel configuration offers additional advantages to the media matric where universal or selective phase change compositions may be transferred and loaded to produce various device configurations. In one application the thermal benefits disclosed may be realized as a cooling effect or heat transmission in a portable and efficient device configuration as energy storage pads for use in furniture, seating and sleeping pads, homes, office, hospitals, assisted living centers, and wheelchairs, automobiles or in garments and footwear. The modular and active channel configuration expands the applicability of this disclosure for highly durable applications such as cooling seats where high pressure and large load may be imposed. The active compartments work in concert to accommodate high impact spots and provide the needed relief without bursting or puncture. The therapeutic benefits are realized in discomfort associated with trapped surplus body heat especially for people with limited mobility and high sensitivity to heat. The positive impact is in reducing skin irritation and skin maceration generally associated with immobility, high skin temperature and the additional moisture which affect skin outer layers and the underlying tissues.

In another embodiment, the configuration may be used to produce an enclosures and protective energy panels to protect labile products during transport and warehouse storage conditions. Various sizes and geometries may be configured and used protective transport packages such as formed boxes, cylinders, envelopes, or pouches and of one or more temperature zones to protect the payload from getting too warm or too cold. This flexibility allows shippers to save on shipping and warehouse cost, reduce inventory demand and reduce waste.

### BRIEF DESCRIPTION OF THE DRAWINGS

FIG. 1 is a schematic view of one design of the progressive chamber thermal unit and a sphere configuration of the present invention. The center chamber is larger than the terminal chambers to allow energy receptor movement from one or both channeled chambers. The terminal and center chambers are connected through a channel opening (0.1-50 mm) along the chamber's adjoining wall. The energy receptor media may be contained in all chambers while spread thinner for maximum surface contact and energy exchange, or contained in any of the chambers for a higher impact. Also illustrated in this figure is a spherical shape may be used to contain the energy storage composition which can be moved within the inner volume when the sphere is applied to odd shaped objects such as a limb or joint for example. The sphere may be partially filled with a gel composition allowing flexibility and enhanced topographical contact with various odd shapes.

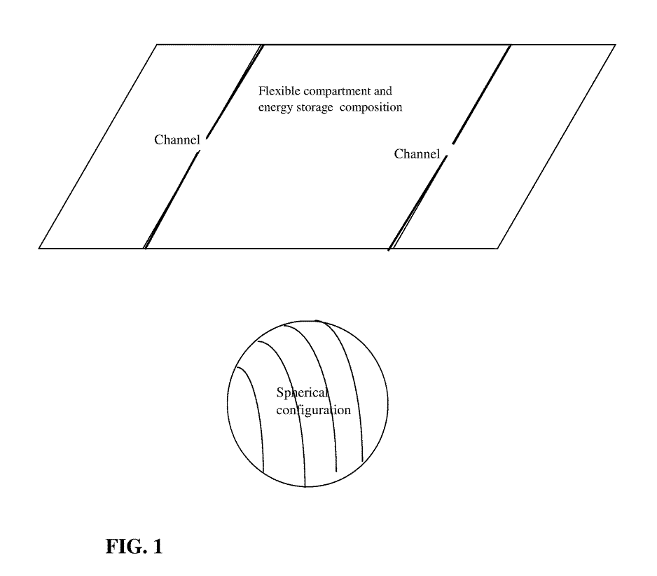

FIG. 2 is a schematic view of one design of the progressive multi-chambers and an inflatable compartment to insulate and provide optimum contact between the device and the treated target area.

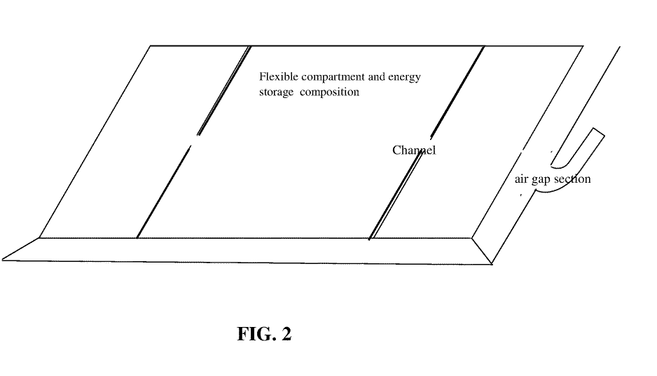

FIG. 3 is a schematic illustration of a support backing and anchoring mechanism. A media matrix with high affinity to energy storage compositions may be laminated to the inner side of the support layer.

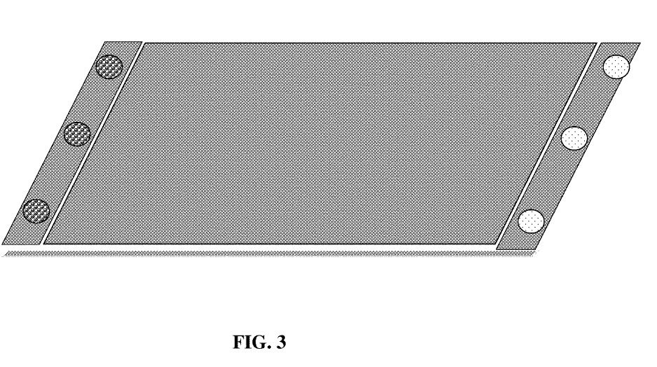

FIG. 4 is a schematic illustration of the progressive multi-chamber unit combined with the support layer and anchoring platform to adhere to a desired user's application area.

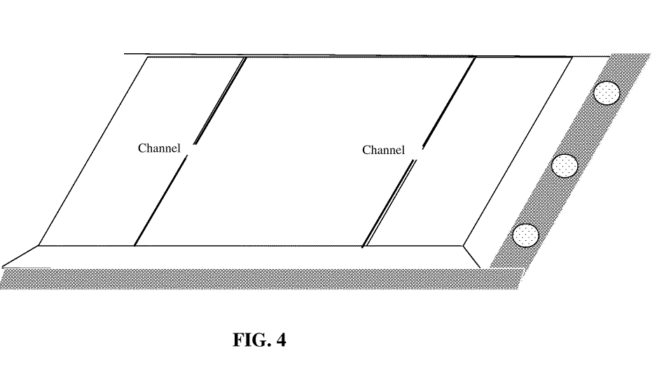

FIG. 5 illustrates another schematic of multi-chamber array and therapeutic energy cell distribution. In this example, we expand the chambers with PCM energy receptor media to be of one or more melting temperatures.

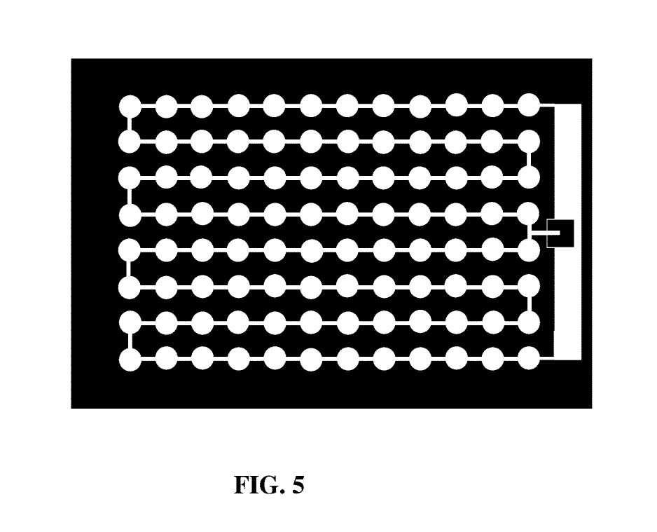

FIG. 6 illustrates the modular design and various temperature zone capabilities in the progressive thermal chambers. In this example, three energy receptors of different temperature are presented as T₁, T₂ and T₃.

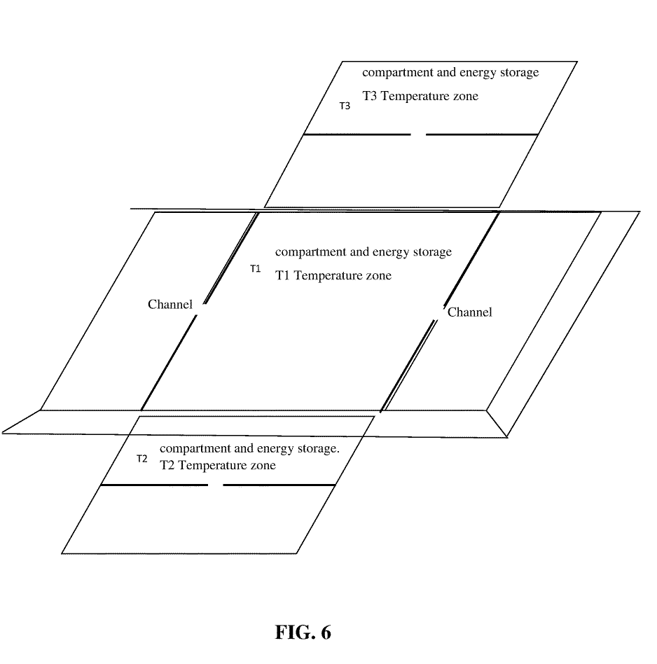

FIG. 7 illustrates the modular design of progressive chambers united with the support layer and the transformability of the device into three-dimensional structure. This example is also configured as ready-to-use and space saving collapsible structure or insert to house and transport temperature sensitive materials such food, medicine, biological samples and organs.

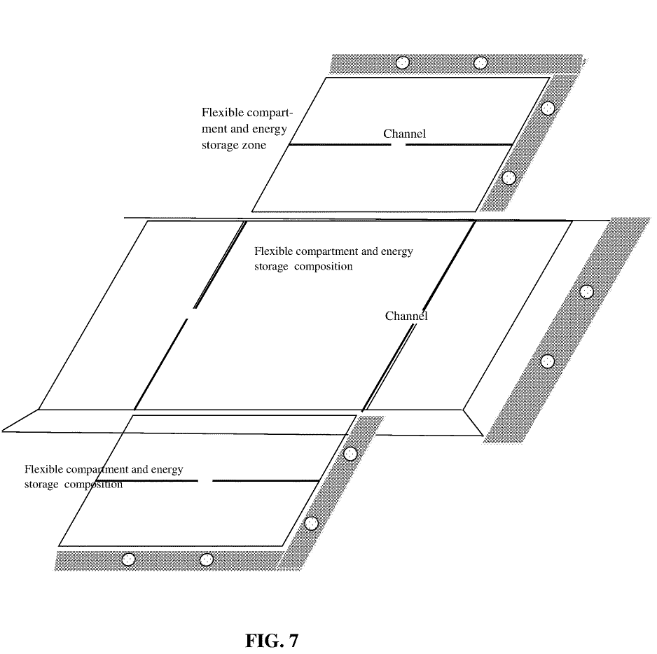

FIG. 8 illustrates a shell design of adjacent progressive chambers forming top and bottom enclosures. Such embodiment forms a sandwich-type enclosure to shield and deliver energy doses for prolonged periods of time. Each energy receptor material composition is held in a closed loop chamber series. This embodiment allows the movement between top level and bottom level chambers.

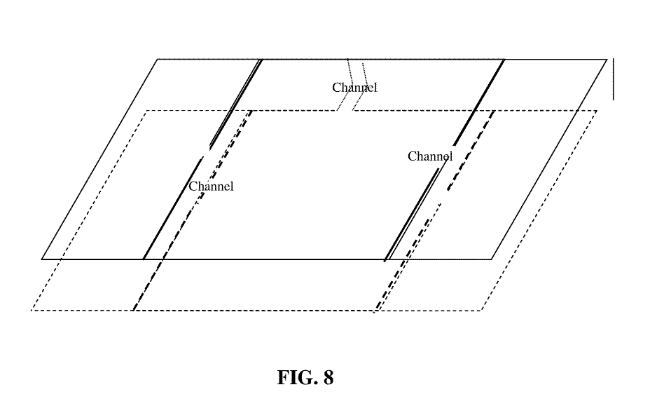

### DETAILED DESCRIPTION

Phase change materials are unique in their ability to transition state and for their high thermal storage capacity. This disclosure describes configurations to produce energy storage products for use in healthcare, transport of temperature sensitive products and therapeutic body temperature management. This invention is generally divided to provide modular, portable energy transfer devices for thermal energy management and therapeutic applications. The principle is generally directed to chambers with heating and cooling capabilities to contain energy storage compositions. It is an enclosure system with one or more flow-through chambers to transport energy storage materials and receptors to fit various payload shapes and sizes.

The Essential Parts of the Device are:
1). the multi chamber unit with interactive channels to contain and move thermal energy receptor materials of one or more temperatures. Various chamber shapes are envisioned including planar and spherical and combination thereof. The material flow is regulated by channel design and material physical properties. Additionally, a porous network media matrix with high absorbing affinity may be integrated in the chambers.
2). the support layer which can be a textile piece or membrane. This membrane in its simplest model can be a reflective coating laminated onto a textile or a thin insulate.
3). A bridging spacer and secondary matrix or gap between the progressive chambers and the support layer. This spacer gap may contain a media matrix absorbent pad to contain and hold energy storage materials or as an inflatable air gap to cushion, insulate or serve as the safety zone to prevent leakage of material.
3). Support and attachments including wraps and sleeves to hold and support the enclosure. These attachments provide anchoring points to desired surfaces for efficient and precise delivery. The anchors may include pressure sensitive adhesives, Velcro, magnetic strips/disks, threaded buttons, clips, and the like. Temperature probes may also be included to monitor the temperature and indicate energy delivery status.
It should be recognized that the various embodiments and drawings are merely described for illustrative fulfillment of the various objectives and principles of the present invention.

In one embodiment, is disclosed the thermal energy receptor being a gel material moved between chambers through a channel (0.1 mm-50 mm) along the chamber separating walls. The PCM thermal energy receptor holding device includes two or more chambers and sub-chambers formed as one unit and may provide one or more varying temperature zones. This dual temperature capability provides a cool area to prevent labile products from overheating as well as preventing the product from freezing or becoming deactivated.

According to one aspect of the invention the progressive flow device is in the form of a pad with multiple chambers on boards and of various lengths and thicknesses. It will also provide a method of attachment of the device to a multitude of surfaces and creation of various enclosure geometries such as pad inserts, boxes, envelopes and pouches for containment and efficient energy exchange.

Another embodiment would include one or more chambers with different energy receptor capacity as exchangers to deliver cold or hot zones to desired areas for any contact time. The design is flexible to accept PCM energy receptors in the form of liquid, gel, paste, powder, crystals or small particles. Such an energized pad may be used for example, as a body cooling cushion or sleeping pad to absorb and release surplus body heat and regulate body temperature.

In another embodiment, an integrated energy-holding device is attached to a flexible support, such that it enables configuration for various shapes or to be attached to different surfaces. The support layer may include an insulate pad, a reflective layer in one or both directions with attachment fixtures such as magnets, buttons, zippers or combinations thereof. The energy receptor material may be cooled or heated to exchange stored energy. It may also be formatted to structures to absorb and emit energy at time intervals correlating with climate conditions. The support layer may also support an inflatable snug-type air gap section in the device. The inflatable feature allows intimate contact between the energy receptor and a user's affected area, for cushioning and delivering maximum therapeutic dosage. This space may also be filled a soft and absorbing pad to capture released material from the above.

The versatility of the design allows energy containment and exchange for many applications including sports medicine and physical therapy, packaging and transport of temperature sensitive materials such as medicine and perishable products. Furthermore, a larger scale application using the principles of this invention will benefit the energy management and steady temperature control of sports gear, uniforms, tents and camping gear to provide energy savings and comfort.

### EXAMPLE 1

The thermal energy storage composition may be produced from aqueous composition with increasing viscosity. It is then loaded into any of the enclosure configurations including a sphere shape, or flat chambers. The energy storage material may be moved through the channels to provide users a diversified cooling or heating applications. The PCM composition may also be enhanced with absorbent thickener and injected in the sphere configuration for example, then the entire sphere is immersed in hot water bath at 70-80 C for a few minutes to activate the composition into a thick gel form. The partially filled sphere then becomes a universal energy pad to fold over any odd shape or limb.

### EXAMPLE2

This example represents a general illustration for the integration of phase change material loaded media matrix with the progressive enclosures disclosed in this invention. For body temperature management, the preferred transition temperature ranges from 18-49° C. A composition is selected from crystalline salt or molten solution, fatty alcohols or esters, fatty acids or aliphatic carbon material. A more specific example may use crystalline salt hydrate such as calcium chloride crystalline salt, sodium sulfate crystalline salt, magnesium chloride or mixture thereof. Other compositions include methyl palmitate, methyl laurate, capric acid, lauric acid, lauryl alcohol, myristyl alcohol, cetyl alcohol or eutectic mixtures. The composition is prepared at temperatures above their melting temperatures. While stirring, an additive selected from aluminum, iron, copper, carbon black, metal oxides, silica particles or graphene Magnesium stearate and calcium carbonate is added to the mixture in amounts up to 10 percent by weight. The composition is then incorporated into dynamic chambers or media matrix and allowed to be absorbed and distributed to produce the PCM thermal energy storage device to store and release heat.

### EXAMPLE 3

In this example an active volume energy storage sphere is illustrated. The geometric flexibility of the shape enables excellent contact with variously shaped body parts or payloads. The media matrix is formed inside the enclosure before energy receptors are loaded. A composition of a holding matrix including absorbent components may be injected into a preformed sphere shape. The PCM energy storage receptor including water, water-based compositions or a phase change material from example 2 is introduced into the holding matrix.

### REFERENCES CITED

U.S. PATENT DOCUMENTS

| | | |
|---|---|---|
| U.S. Pat. No. 5,417,082 | May 1995 | Foster et. al |
| U.S. Pat. No. 5,441,170 | August 1995 | Bane |
| U.S. Pat. No. 6,822,198 B2 | November 2004 | Rix |
| U.S. Pat. No. 7,179,281 B2 | February 2007 | Ferdinand |
| U.S. Pat. No. 7,516,600 | April 2009 | Flora |
| 2013/0255306 A1 | October 2013 | Mayer |
| U.S. Pat. No. 8,600,903 B2 | December 2013 | Eller |
| U.S. Pat. No. 8,226,699 B2 | July 2012 | Evans |

That which is claimed is:

1. A thermal storage apparatus with flexible modular multiplicity that is flat or spherical in shape and communicating channels for the containment and movement of PCM compositions and thermal energy receptor media that can be configured to produce various shapes and geometries from thermal pads and envelopes to enclosures.

2. The device of claim 1 further comprising a support media matrix layer capable of containing another energy storage material and as a high affinity absorption media matrix including cellulosic and synthetic fiber network.

3. The device of claim 1 wherein the modular chambers are serially connected or in parallel and where some or all chambers are connected.

4. The device of claim 1 wherein energy storage composition material is of one temperature or of various temperatures.

5. The design of claim 1-4 combined to generate a therapeutic mat for body temperature management configured for joint pain management, use as a seating cushion, back support or sleeping pads.

6. The device of claim 1 wherein in the second layer comprises an insulating support with fasteners enabling the attachment of the device to surfaces including three dimensional objects.

7. The system in claim 1-6 wherein a three dimensional structure may be formed to house and protect temperature sensitive products during storage and shipment.

8. The device of claim 1-7 wherein temperature indicators or energy load levels are integrated within to monitor energy storage status and target temperature.

9. The device of claim 1-6 wherein a sealed air gap spacer is created next to the second support layer.

10. The device of claim 1 wherein the PCM and energy receptor material comprises of liquid to highly viscous organic or inorganic PCM, aqueous material composition including water miscible materials, fatty acid/esters, fatty alcohol, or aliphatic carbons.

11. The device of claim 1-10 may be configured as an insert panels for storage or shipping containers as well as stand-alone energy enclosure.
